(19) United States
(12) Patent Application Publication
(10) Pub. No.: US 2011/0081333 A1
(43) Pub. Date: Apr. 7, 2011

(54) APPARATUS AND SYSTEM FOR TREATMENT AND PREVENTION OF BAGS UNDER EYES

(76) Inventors: Totada R. Shantha, McDonough, GA (US); Jessica Gowramma Shantha, McDonough, GA (US); Lauren Asha Shantha, McDonough, GA (US); Usha Shantha Martin, Macon, GA (US)

(21) Appl. No.: 12/964,755

(22) Filed: Dec. 10, 2010

(51) Int. Cl. G02C 11/00; A61K 36/82; A61K 38/46; A61K 31/568; A61K 31/565; A61K 38/18; A61P 17/00; A61N 1/00

(52) U.S. Cl. 424/94.62; 351/158; 424/729; 514/179; 514/182; 514/7.6; 607/141

(57) ABSTRACT

A simple, safe and effective invention devices for treating bags under the eyes pressure exerting glasses devices, compress badges, bag pressure adhesive strips, cosmetic pressure adhesive strips; to be put on to prevent, and to reduce the bags under the eyes. They are designed to apply antiwrinkle and other cosmetic and therapeutic agents such as hormones, known therapeutic agents, as well as other pharmaceutical, biochemical, nurticeuticals and biological agents, or compounds to enhance the health and beauty of the eye, strengthen the lower eye lids and to treat many oculopathies which can contribute to the condition. They can be used for the eye makeup cosmetically as adjuvant to eye makeup. The electrical stimulation and electromotive force are incorporated in the invention to strengthen the lower eye lids muscles, to improve the health of the eye lids skin and for the delivery of therapeutic agents to treat various oculopathies.

### CROSS REFERENCE TO RELATED APPLICATIONS

This invention is particularly designed to compress through physical pressure or force, to prevent or treat bags under eyes (BUE). The treatment of BUE would be with lower eye glass devices, eye bandages, eye lid adhesive strips, electrical stimulation with the bag under eye adhesive strips, and cosmetic application all of which reduce and/or eliminate the bags under the eyes, mild swelling, or puffiness under the lower eye lids which are common with advancing age. This invention can be used to prevent the condition of bags under the eyes due to lack of sleep, alcoholism, allergic conditions, nutritional factors, after surgery of the eyes, and oculopathies, due to heart, kidney, endocrine and other systemic diseases. They can be provided with therapeutic agents to treat eye lid diseases and other oculopathies. In addition, they can be used to enhance the cosmetic make up.

### BACKGROUND OF THE INVENTION

Bags under the eyes (BUE) encircle the lower lids in a semicircular crescent outline extending to the rim of the orbital ridge of the maxillary bone from medial to lateral canthus of the eye due to swelling or puffiness under the lower lids of the eyes which are common with advancing age (FIG. 1, 6). The bags under the eyes are made up of fat with tissue fluid intertwined with reticular fine network of connective tissue and capillary network.

The cause of this condition is simple. With aging, the tissues around the eyes (especially the lower lids), including the muscles, connective tissue, ligaments, orbital septum, and skin supporting the eyelids weaken. Due to this weakness, the normal fat that helps support and encircle the eye balls in the orbital cavity and lower part of the eye ball migrate forward and downwards into the lower eyelids which causes the lids to appear puffy with semicircular swelling.

Some tissue fluid also accumulates in the space below the eyes in front of the maxillary bone due to gravitational effects adding to swollen bags under eyes. The bags under the eyes increase in size as the evening encroaches which explains the effect of gravity and erect posture. Various systemic diseases of the heart, lungs, kidney, and host of others with fluid accumulation can contribute to the condition as a secondary effect. The bags under the eyes are usually a cosmetic concern and sometimes can be a sign of a serious underlying medical condition. Home remedies like cold compresses, wet cold tea bags, can help improve the appearance of bags under the eyes. Applying cucumbers slices to the eyes is unproven but outwardly a safe therapy.

For persistent bags under eyes, cosmetic surgical treatments (blepharoplasty) are available and expensive. Our invention will help those who do not want to undergo surgery, or cannot afford surgery.

Symptoms of bags under the eyes can include: Mild to moderate crescentic swelling, saggy or loose skin, wrinkles in the skin, and dark circles. The bags under the eyes are safe harmless innocuous condition which doesn't require medical care.

A person can see a physician if the swelling is severe and persistent. A specialist physician will want to rule out other causes of the bagginess under the eyes other than aging that can contribute to the bags under the eyes, such as heart, lung, liver, kidney, thyroid problems, or etc. Also poor nutrition, sleep deprivation, excess alcohol consumption, water retention due to more water drinking, or eating salty foods need to be ruled out before considering expensive physician consultations and surgery.

Etiology: As we age, the skin, the tissue (collagen and elastic tissue) under the skin and the muscles which make up the eyelids weaken. The skin begins to sag and the fat that is confined to the area around the eye (lower half of the eyeball and orbit) migrates forward and downwards below the eyes (FIG. 1, 6). The space below the eye balls (infra orbital space) accumulates fluid, making the under-eye area appear puffy or swollen. Several factors can lead to the condition: Fluid retention due to changes in weather (for example, hot, humid days), hormone levels or eating salty foods, sleeping prone, not getting enough sleep, allergies or dermatitis, when the puffiness is accompanied by redness and itching and heredity factors can play role.

Before one decides to take measures against the bags under the eyes, the person needs to know the cause of the symptoms. Is the condition temporary or appears and disappears or is chronic? What would be the best course of action? What treatment approach do you consider or recommend, if any? What will be the treatments cost? Does medical insurance cover these costs? What results can one expect? Is there anything a person can do at home to improve the symptoms? What kind of follow-up, if any, should one expect? (From MayoClinic.com web site http://www.mayoclinic.com/health/bags-under-eyes/DS01151).

Studies show that with aging, there is a significant increase in anterior inferior periocular soft-tissue volume and that the fat expansion is the main contributor to this volume increase (bags under eyes). These studies provide supporting evidence that orbital fat expansion occurs with age which is the primary age-associated contributor to lower eyelid prominence, rather than globe descent or fat repositioning caused by weakening of the orbital septum or loose lax skin (Darcy, Sean J. M.D.; Miller, Timothy A. M.D.; Goldberg, Robert A. M.D.; Villablanca, J Pablo M.D.; Demer, Joseph L. M.D., Ph.D.; Rudkin, George H. M.D. Magnetic Resonance Imaging Characterization of Orbital Changes with Age and Associated Contributions to Lower Eyelid Prominence. Plastic & Reconstructive Surgery: September 2008-Volume 122-Issue 3-pp 921-929). Our studies do show that the fat migrating from the retro bulbar space and the space between the optic nerve and the muscle cone migration is responsible for increase in this fat besides biological or pathological increase in the fat supporting these studies. Our invention will push back the fat to the orbital cavity which has enough tissue space to accommodate the repositioned expanding fat.

Before resorting to drastic surgery, try the following guidelines that can help to reduce or eliminate bags under the eyes: Wet a clean wash cloth with cool water. While sitting up, apply the damp washcloth to the skin around eyes for a few minutes using mild pressure in the sitting position, get 7-8 hours of sleep (sleep with head slightly raised on a comfortable pillow), avoid allergens such as under the eye hair dyes, soaps, cosmetics or other allergens, use anti allergic medication if there is an allergic condition, and consult an ophthalmologist if needed.

Apply cold tea bags which can help and slow the aging skin due to its antioxidant catechin contents such as Epigallocatechin (EGCG). Avoid rubbing the eyes often. Reducing the weight and quitting smoking can help. Exercises such as tightening the jaw with a smile-angle of the mouth drawn outward; lift the weight of your finger using lower lids without the movement of the eyebrows for a minute or two twice a day, looking up and down with both eye lids closed without moving the eye brows can improve the conditions.

Medical treatments are available if you're concerned about the appearance of under eye swelling advancing and related to other systemic diseases. Treatment may not be covered by medical insurance if it's done exclusively to enhance appearance (cosmetic intervention). Skin treatments done to treat wrinkles, such as laser, resurfacing or chemical peels, may improve skin tone and tighten the skin which may reduce the under-eye swelling and improve discoloration to some extent. Eyelid surgery (blepharoplasty) is an alternative to remove bags under eyes.

U.S. Pat. No. 7,603,723 B2 A discloses a mask that includes a flexible backing and an expandable membrane configured to overlay the eyes and nose of a wearer. This does not address the eye bags as described in this invention.

U.S. Pat. No. 6,623,517 B I discloses a device to cover the eyes completely with a convex gelatinous device. This does not deal with the baggy eyes of the lower lids and the device completely obstructs the vision and disables when in use.

There is no specific invention which treats the bags under the eyes specifically which our invention provides.

### SUMMARY OF THE INVENTION

This invention relates to the bags under the eyes (BUE) compressor adhesive strips with or without eye makeup provisions, compressor eye badges, and eye glasses to wear or to put on the eyes to prevent or reduce the bags under the eye.

It is the goal of this invention of compressor for bags under eyes to use it at day or night-time, watching the television, and other activities which this will prophylactically prevent and treat bags under the eyes.

It is the intent of this invention to be used to treat eye and eye lid afflictions (oculopathies) including wrinkles, black circles around the lids, and to deliver therapeutic agents.

It is another objective of this compressor invention to be used for bags under eyes also to be used to enhance the makeup of the eyes and to reduce the dark spots around the eyes with cosmetic agents at the same time reduces or prevents the development of the bags under the eyes.

It is another object of this invention to stimulate the orbicularis oculi muscle, connective tissue of the eye lid and skin covering the eye lids with mild direct electrical current to strengthen these structures, and increase the thickness of the skin of the lower eye lid to hold back the fat in the infra orbital space and prevent bulging.

It is another object of this invention to incorporate various known therapeutic agents, as well as other pharmaceutical, biochemical, nurticeuticals, and biological agents or compounds to treat various oculopathies affecting the eye lids at the same time stimulate the orbicularis oculi muscle, connective tissue, and skin of the lower lid involved in the bags under eyes development.

It is another object of this invention to incorporate various known eye makeup cosmetic applications with or without therapeutic agents, as well as other pharmaceutical, biochemical, nurticeuticals, and biological agents or compounds to treat various oculopathies to enhance the beauty of the eye.

Other features and advantages of the instant invention will become apparent from the following description of the invention which refers to the accompanying drawings.

### BRIEF DESCRIPTION OF THE DRAWINGS

FIG. 1 is a sagittal section of the eye ball in the boney socket 700 shows the various histological structures involved in Bags under eyes development.

FIG. 2 is a diagram of the eye glass 100 showing its various components which make up this eye wear.

FIG. 3 is a perspective view of the preferred embodiment 200 showing the eye glasses type invention to be worn to prevent or to reduce the bags under the eyes of the lower eye lids.

FIG. 4 is a perspective view of the preferred embodiment 700 showing the eye glasses invention which was described in FIG. 3. In addition it contains the reading or prescription glasses 609.

FIG. 5 is a perspective view of the preferred embodiment 300 similar to FIG. 3, showing the eye glasses type invention to be worn to prevent or to reduce the bags under eyes.

FIG. 6 is a perspective view of the preferred embodiment 400 similar to FIGS. 4 and 5 showing the eye glasses type invention to be worn to prevent or to reduce the bags under eyes.

FIG. 7 is a perspective view of the preferred embodiment 500 showing our invention to wear to prevent the bags under the eyes.

FIG. 8 is a diagram showing the lower lid bag eye badge preventing badge 600 in position.

FIG. 9 is the frontal view of the eye ball in the boney socket 800 made up of maxilla, zygomatic, and frontal bones which they form the edge of the boney eye socket external borders 801.

FIG. 10 is the frontal view of the lower lid eye elastic adhesive strip 900 placed on the lower eye 901.

FIG. 11 is the frontal view of the lower lid eye adhesive strip 1000 to be placed on the lower eye lid on the site of the bags under the eyes.

FIG. 12 is the top frontal view of the lower lid bags under the eye adhesive strip 2000 similar to 1000 to be placed on the lower eye lids on the site of bags under eyes.

FIG. 13 is the top frontal view of the lower lid eye adhesive strip 3000 similar to 1000 to be placed on the lower eye lids on the site of bags under eyes with direct electrical stimulator.

FIG. 14 is a diagram of the bags under the eye adhesive strips which can be applied as make up adhesives 4000 in addition.

FIG. 15 is diagram of the bags under the eyes where the eye lids can be applied as make up adhesives 5000 in place with the eye lashes liner make up.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

As shown in the drawings for purposes of illustration, the present invention relates to bags under eyes badge compress, elastic compressor of lid adhesive strip, eye glass compress, and eye glasses device to be put on for preventing and treating bags under the eyes which are common as a person ages.

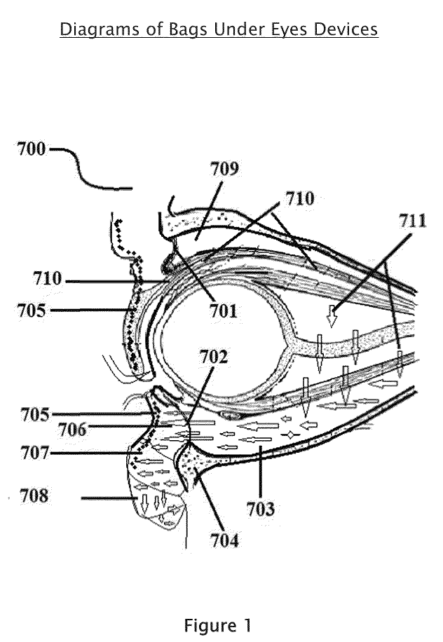

FIG. 1 is the sagittal section of the eye ball in the boney socket 700 shows the various histological structures involved with various types, severity and stages of bags under eyes (BUE) development 706, 707 and 708. Our studies show that the etiology of development is due to its histological structural changes related to aging and gravity. In the young, the eye ball is surrounded by the periorbital fat contained within the boney orbit suspended and contained by the orbital septum 701, 702.

The upper eye lid and its fat in superior orbital space 709 are held back from bulging due to thick superior orbital septum 701, levator palpebrae superioris and the large extensive tarsal plate. Besides, the levator palpebrae superioris muscle 710, which strengthens the upper eye lid and constantly pushes back the content of the superior orbital space (periorbital fat) which prevents the baggy bulging of the upper eye lids. This thickened part of this muscle which comes closer to the tarsal plate and eye lids, that adds strength to the superior orbital septum is lacking in the lower lids.

This muscle is absent in the lower lids and has a small tarsal plate, which further weakens the wall of the lower lids allowing the inferior periorbital fat migrate and bulge easily as Bags under eyes. The inferior orbital septum 702 is attached to the edge of boney orbit 704 (maxilla) that it is thin and weak compared to the superior orbital septum 701 which is thick and short. With age, the skin of the lower eye lid with its orbicularis oculi muscle 705 loses their elasticity and tonicity due to degenerative changes in the underlying collagen and elastic tissue. The fat under the inferior part of the eye ball increases and the edematous fluid easily accumulates in this fat connective tissue compartment due to gravitation pull (dependent part).

The fat content of this space contributes from the gradual swift, migration, and gravitational pull of the fat found in the space within the cone of muscles behind the eye ball 711 in the retro bulbar space and surrounding the optic nerve. This fat communicates with the infra orbital fat. When the bulk of fat increases, the inferior orbital space 703 can no longer hold it. The inferior orbital septum 702 becomes weak with aging, and loses tensile strength.

Hence, the content of the inferior orbital space 703 protrudes at this line of least resistance (arrows), i.e. anteriorly, inferiorly through the weakened inferior orbital septum 702 and orbicularis oculi muscle and skin 705 which appears as bags under eyes 706-708. The bags under eyes condition can be mild 706, moderate 707 and severe 708 with over hang on the edge of the boney maxilla. The fat that accumulates under the lower eye lid as bulge is soft, semi liquid and movable which can be easily pushed back to inferior orbital surface or pocket 703 that reappears when the pressure is removed.

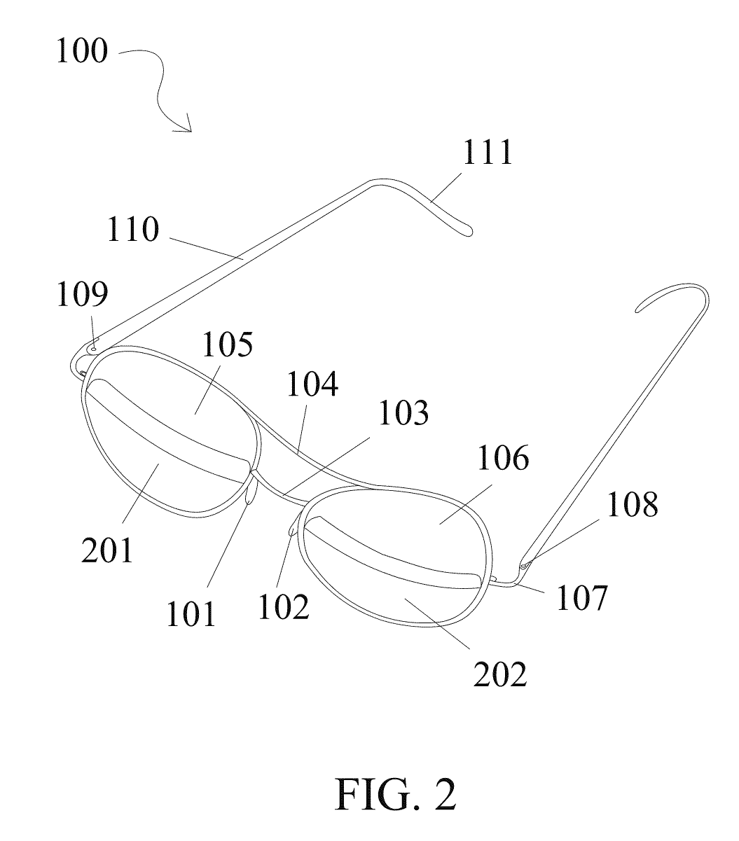

FIG. 2 is the diagram of the eye glass compress 100 showing its various components which make up the eye wear that we describe. It is presented here to show our inventions wearing as an eye glass to help prevent or to push back bags under eyes. It is worn over the eye to prevent or to reduce the eye bags that develop. The eye glasses are composed of nose pads 101, nose pad arms 102, a saddle bridge over the nose 103 that distributes the weight evenly and at the same time holds it in position.

Double bridge has a reinforcing bar 104 over the top of the bridge. The stable bridge 103 gives more stability to the glasses while adding esthetic value. The lenses 105 are encased in wires/rimless frame 106 with end pieces 107 extending from the frame front which have screws 108 and hinges 109 to be attached with the temple arms 110, and the temple tip 111 with plastic coating behind or over the ears to hold the eye wear in place without falling during movements of the head which make them comfortable to wear. A pair of pockets 202 provide the compression.

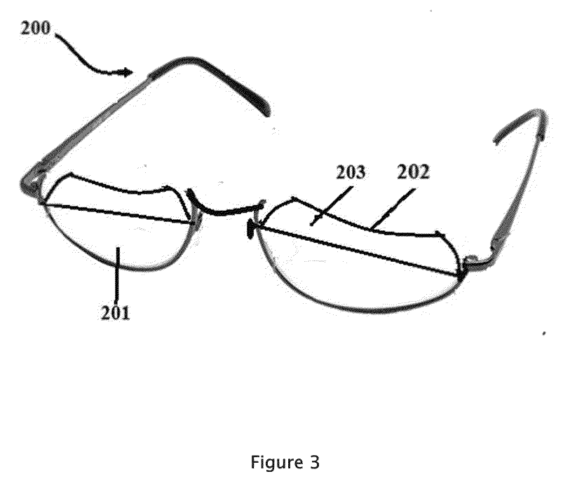

FIG. 3 is a perspective view of the preferred embodiment 200 showing the eye glasses type compressor invention to be worn to prevent or to reduce the bags under the eyes. It is designed in a way that it will allow the vision and will fit on the bag under the eyes to prevent their prominence and protrusion. They are composed of semicircular plastic or flat glass 201 which can be flat, convex or concave, situated in the lower half frame similar to reading type glasses without obstructing the vision.

On the back the glasses facing the lower rim of the eye opening, which has an semicircular crescentic shaped elastic or malleable silicone, gauge or cotton pocket 202 which accommodates silicon, cotton filler, gel or inflatable balloon 203 to press against the lower lid without interfering with the vision. When the filler material is inserted into the pocket 203 it takes the curvature 202 of the lower eye lids bags under eyes and the maxillary border of the eye socket to exert even compression/pressure on the baggy lower eye margins and on the maxillary bone without obstructing the vision when one wears these glasses.

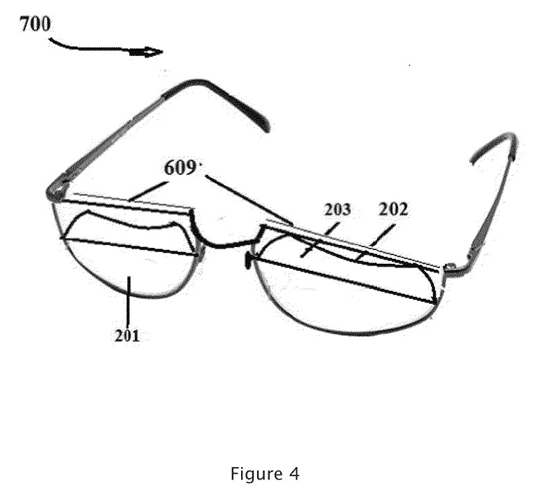

FIG. 4 is a perspective view of the preferred embodiment 700 showing the eye glasses compressor invention used to treat bags under eyes as described in FIG. 3. In addition this contains the reading or prescription glasses 609 added to wear while in bed reading, on the sofa, or while working in the house and at the same time and prevents, cures or curtails the bags under the eyes.

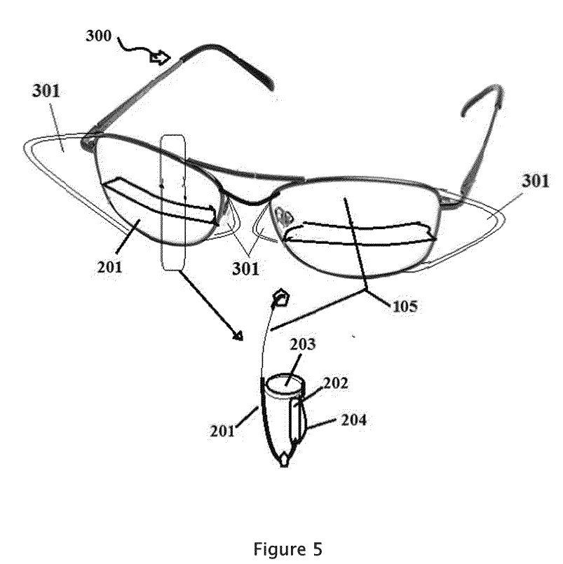

FIG. 5, is a perspective view of the preferred embodiment 300 similar to FIG. 3, showing the eye glasses. It incorporates the prescription eye glasses 105 or sunglasses in the full frame that can be used during the reading or driving activities and other uses of the eye sight that are not interrupted. Note the bulge 204 in the posterior elastic surface membrane 202 presses against the eye bags due to a filler 203 under the eye and prevents their further progression. The square shows the cut section view of the glass of our invention.

The pocket 203 will accommodate filler material such as an inflatable balloon, cotton gauge wad, synthetic material, silicone, gels, and other non-allergic, non-reacting, nontoxic, non-coloring material which is replaced as needed. These glasses can be provided with membrane shields 301 around the frame to retain the moisture in additions to prevent dry eye syndrome. These shields 301 can be made of a transparent plastic. The compress device pockets can be equipped so that sterile wet wad of cotton, plastic sponge or any other suitable material be inserted so as increase the moisture around the eyes for the treatment of oculopathies and dry eyes syndrome. The moisture being retained by the shield 301 around the edges of the frame extending around the eyes.

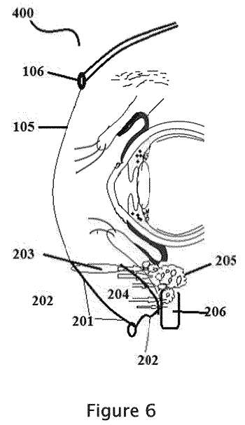

FIG. 6, is a perspective view of the preferred embodiment 400 and is similar to FIGS. 4 and 5 showing the eye glasses type compressor invention to be worn to prevent or to reduce the bags under the eyes. It shows the wearing of our invention to reduce the bags under the eyes (FIG. 6, 205) which are made of mostly fat and some tissue fluid. Arrows shows the pressure on the bags under the lower eye lids of the eyes. Note; that the lower end of these inventions lies in close proximity to the maxillary bone 206 which forms the boney orbit lower surface, that prevents the bulge spreading from the lower eye lids and the eye ball in the orbital socket on to the maxillary bone.

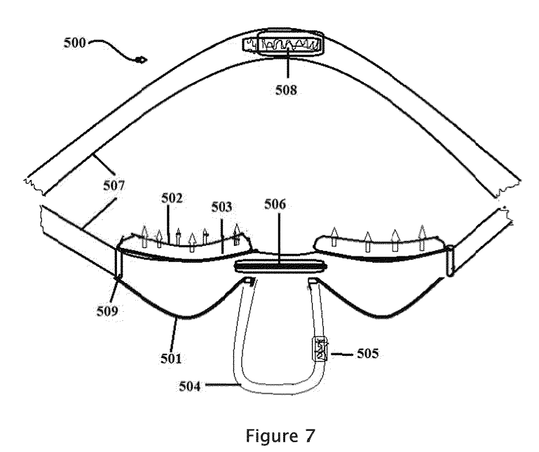

FIG. 7, is a perspective view of the preferred embodiment 500 showing our compress invention badges to wear to prevent the bags under the eyes. The invention is to wear to prevent or to reduce the bags under the eyes. This eye badge invention is made up of molded pliable plastic, metal, thick linen, or other similar synthetic or semi synthetic material.

It is semicircle crescentic shaped outer thick plastic or thick linen layer 501 resembling the bags under the eyes with a flexible pocket 502 facing the lower eye lid and the maxilla. This pocket 503 will accommodate an inflatable balloon or cotton gauge wad, plastic pad, silicone, or malleable plastic gel filled material and other non-allergic, nontoxic, non-reacting filler in the pockets 503. It is shaped crescentic like the lower lid maxillary configuration that it exerts even pressure on the bags under eyes. That means when the filler material is inserted; it curves like a segment of circle to fit the bags under eyes.

The arrows show the direction of the pressure exerted against the crescentic bags under eyes located at the lower lid and the maxilla. It has a bridge 506 that arches up over the nose between the two semicircle eye bags extending close to the medial canthus of the eye. It acts as a saddle bridge which spreads the weight across the sides on the top of the nose and holds the badge 500 in place. It is made up of malleable aluminum or other metal or plastic that can be shaped according to the anatomical configuration of the nose of the user. It prevents sliding of the eye bag wear upwards and keeps at the desired position where it applies inward pressure at the lower lid.

From the inner side close to the nose, there is a rubber, elastic, or flat flannel string 504 with Velcro 505 that can be adjusted so that it can circle around the lower end of the nose and hold the bags under eyes badge wear in position which prevents its sliding on the lower lid and covering the visual field of the eyes or scratches the cornea or the conjunctiva.

It can be worn all night without displacement on the face or the eye ball due to tossing and turning of the head during sleep. The invention has two elastic bands, less than ¼ of an inch wide 507 with hook and loop fastener on the back 508 to hold the bags under eyes badge bag wear in position. It is attached to the ends of the badge 509 by staples, stitches, adhesive material, or buckle when this inventive bags under the eyes device is worn which is known as bags under eyes preventive design badge.

It is positioned so that it will not slide up and cover only the lowest part of the lower eye lids and the maxilla. To hold the badge in position, it can be broad having an upper layer with eye hole attaching to the eye brows and frontal area of the forehead close to the eye brows.

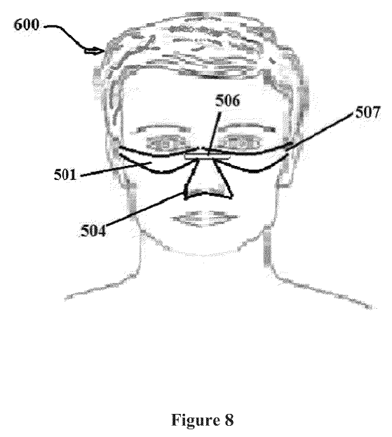

FIG. 8, is a diagram showing the bags under eyes lower lid bag eye badge compress 600 in position. It shows the eye lid bag preventing cups 501 with nose securing elastic band 504 around the nose. It has a bridge 506 that arches up over the nose between the two semicircle eye badges 501 that is malleable and adjusted to bring the nasal ends of the cups closer to the area of formation of baggy eyes. The invention shows two elastic bands less than ¼ of an inch wide 507 with hook and loop fastener cross over the top of the ear all the way to the back to secure the bags under eyes badge compressor and to hold it in position.

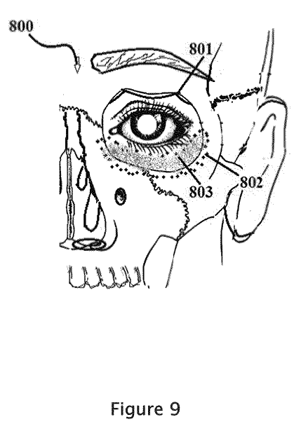

FIG. 9 is the frontal view of the eye ball in the boney socket 800 made up of maxilla, zygomatic, and frontal bones. They form the edge of the eye socket external border 801. The dots at the lower lid 802 show the site and extent of the bags under eyes 803 location and the use of our inventions at this anatomical location.

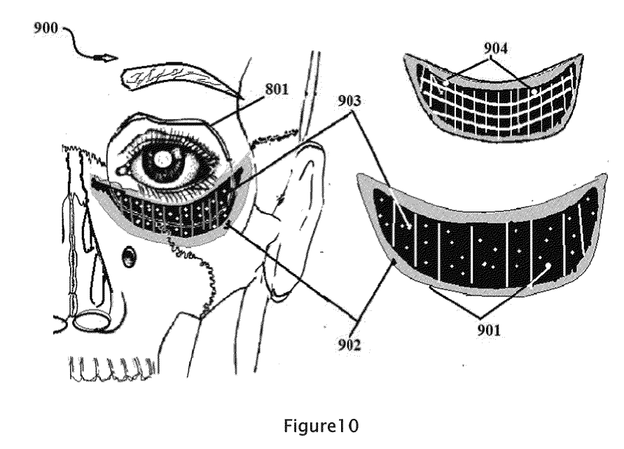

FIG. 10 is the frontal view of the lower lid bags under eyes compress/pressure elastic adhesive strip 900 placed on the lower eye lid 901 on the site of bags under eyes located on eye socket external border 801. It is be made of elastic natural, synthetic plastic, silicone, or such material with pores 903 that can be stretched and attached to the site of the bags under the eyes area to help prevent the development, reduce bulge, and to drive them back to the eye socket.

The edges of the bags under eyes adhesive strip 901 is attached to the edges of the skin with an adhesive thin plastic transparent strip 902 which will hold the bags under the eyes adhesive strip in position under pressure to prevent the bags from developing under the eyes. It is applied to the lower eye lids with slight stretch that the elasticity exerts light pressure on the baggy part of the lower eye lids and pushes it back to the boney socket.

It can be porous (white dots), to allow ventilation of the skin and leak the sweat. They can be made up of transparent material to reflect the color of the eye lid skin on which they are applied. The Bags under eyes adhesive strip can be made like a fish net 904 which allows the application of eye make up at the same time prevent the Bags under eyes bulging by pressure exertion on them.

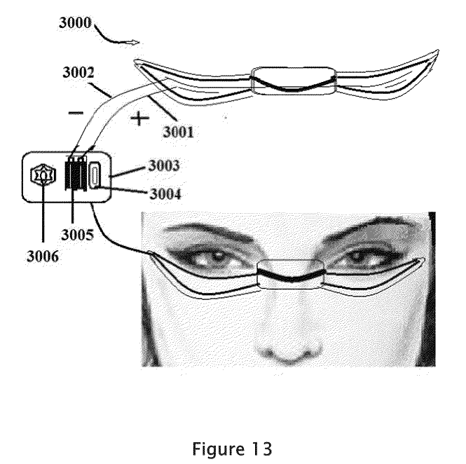

It is colored to match the eyes and eye makeup. The natural eye makeup can be applied on these bags under eyes adhesive strip. The adhesive is attached at the edges or the entire surface of the lower eye lid. The undersurface of the bags under eyes adhesive strip can be coated with various known therapeutic agents, as well as other pharmaceutical, biochemical, nurticeuticals, and biological agents or compounds to improve the skin health, which include anti-wrinkle agents, hormones, anti-allergic agents, antimicrobial agents, and other therapeutic/cosmetic agents while in use to prevent the development of bags under the eyes and to treat various oculopathies. It can also have electrical stimulator wire connected as described in FIG. 13 to stimulate the lower lid muscle, connective tissue and skin so as to strengthen them and prevent the development or reduce the bags under eyes. The bags under eyes adhesive strip can be worn all day and night which are colored to give natural skin color of the eye lids.

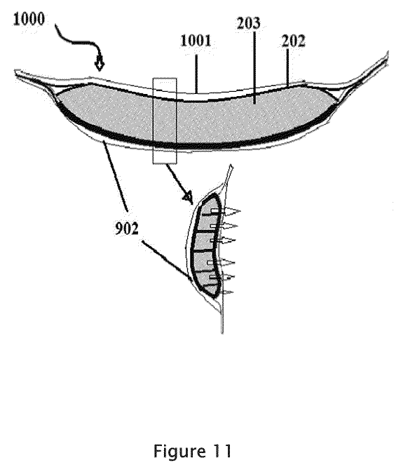

FIG. 11 is the frontal view of the lower lid of the eye compress where the eye lid bag adhesive strip 1000 is to be placed on the lower eye lid on the site of bags under the eyes. It is similar to the one described in FIG. 10 except this adhesive strip has a silicone or cotton or synthetic or semi synthetic material padding conforming to the crescentic shape of the bags under the eyes.

When applied, the padding exerts slight inward pressure (arrows) against the lower eye ball, pushing the baggy bulging inside the eye-boney socket (arrows). The edges of the bags adhesive strip 902 is attached to the edges of the skin with an adhesive thin plastic strip which will hold the adhesive strip in position under pressure to prevent the bags under eyes development. It is applied to the lower eye lids with slight stretch that the elasticity exerts light pressure on the protruding eye bag.

A stretch applicator forceps can be provided to facilitate the applications of this adhesive strip type compressor on the lower eye lids as well as the upper eye lids. It can be porous to allow ventilation of the skin and is colored to match the eyes make up and eye skin color. The adhesive is attached at the edges or the entire surface of the lower eye lid 902. The undersurface of the bag adhesive strip 1001 can coated with therapeutic agents to improve the skin health, antiwrinkle agents, hormones and other therapeutic and the exposed surface can be colored or coated with cosmetic agents while in use to prevent the development of bags under the eyes at the same time improve the underlying skin and its structure and treat various oculopathies.

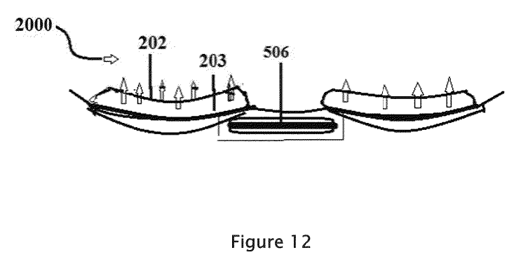

FIG. 12 is the top frontal view of the bags under eyes compress of lower lid eye adhesive strip 2000 similar to 1000 to be placed on the lower eye lids on the site of the bags under the eyes. It is similar to the one described in FIG. 11, except, this adhesive strip has a silicone or cotton or synthetic material padding which conforms to the shape of the bags under the eyes.

When applied, the padding exerts slight inward pressure (arrows) against the lower eye ball which pushes the baggy bulging inside the eye socket (arrows). It has a bridge 506 that arches up over the nose between the two semicircle eye bag adhesive strips extending close to the medial canthus of the eye. It acts as a saddle bridge which spreads the weight across the sides on the top of the nose and holds the badge 2000 in place. The spring action presses the metal nasal bridge plate which exerts the tension on the lower eye lid and pushes the bags under the eyes back into the inferior surface of the orbital cavity as shown in FIG. 1 as 703 and FIG. 6 as 205.

FIG. 13 is the frontal view of the lower lid bags under eyes adhesive strip compress 3000 similar to 1000 to be placed on the lower eye lids on the site of bags under eyes joined on the nose bridge equipped and incorporated with orbicularis oculi muscle and skin stimulation by electrical force 3003 is attached.

The electrical wires run on the undersurface of the lower lid adhesive strip and are connected to the electrical control box 3003 by electrical positive 3001 and negative 3002 wires. The electrical control box 3003 is equipped with on and off switch 3004. The battery package 3005 connected to the wires under the eye lid adhesive strip, and current flow control switch 3006 which delivers the desired adjustable comfortable levels of current in milliamps (MA) so as to initiate mild contraction of the muscle fibers of the lower lid.

The electrical current delivery adjuster switch 3006 will deliver desired level of milliamps of electricity without undue discomfort of the user. The user can adjust by increasing or decreasing the electrical stimulation delivered to the lower lid (and/or upper lid) skin and muscle to their desired tolerable levels using this control mechanism 306. The electrical stimulation will increase and maintain the tone of the musculature at the same time increase the size and strength of the muscle due to repeated contraction—relaxation provides by the milliamps of electrical motive force controlled by the electricity delivery switch 3006.

The electrical current (force) also stimulates the division of the basal cells and the keratinocytes of the epidermis and fibroblasts of the dermis which increases the strength and the thickness of the lower lid (and/or upper eye lid) skin. The increased muscle tone, strength of the orbicularis ocularis muscles with increased thickness of the skin of the lower lid and increased connective tissue of the dermis will prevent the fat and tissue fluid from the under surface of the eye ball and escape of fat and fluid will present as bags under the eyes.

This electrical stimulator can be incorporated into all of our inventive devices we have described herein. The figure below shows how it is worn on the eyes on the face to prevent bags under the eyes which the control box is carried with long wires in the users' shirt or coat pocket. The control box can be connected or disconnected as desired by attaching or detaching the electrical connection from the control box battery pack.

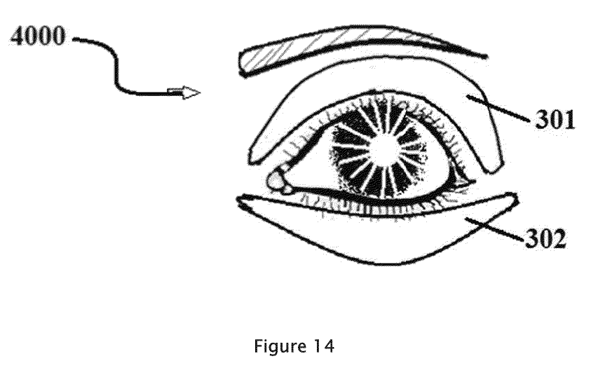

FIG. 14 is diagram of the eye lid make up adhesives 4000 in place. The upper eye lid cover 301 can be of shape of the eye lid or can be larger to be cut and to fit the eye lids. The same is true of the lower eye lid adhesive strip 302, except, it is elastic to exert slight pressure at the junction between the eye ball and the boney orbit which prevents bulging and the development of the bags under the eyes.

Both 301 and 302 can be of different color to match the cosmetic eye makeup. They are porous to allow the sweat to leach out and will also take the makeup applied on its external surface. They will take any cosmetic eye lid make up to match the eye lids or preferred by the person. The lower lid eye lid make up adhesive is elastic so as exert tension on the skin of the lower lid and prevent the bulging of the bags under eyes.

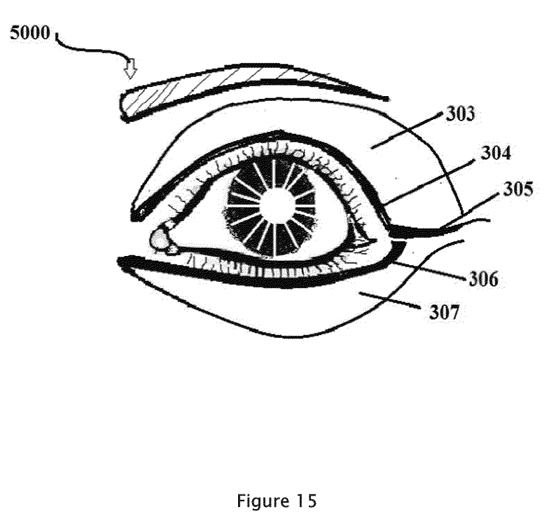

FIG. 15 is diagram of the eye lids make up adhesives 5000 in place. The upper eye lid cover 303 can be the shape of the eye lid, and can be larger to be cut and to fit the eye lids. The edge of this upper eye lid has dark eye lash liner marker 304 which extends laterally from the external lateral canthus in a curved fashion to suite the eye makeup of the lid margins with ease.

The lower eye lid adhesive strip cover 307 is the same as the upper eye lid and has more elasticity that it can exert pressure at the junction between the eye ball and the boney orbit to prevent the development of the bags under the lower lid. It has dark eye lid liner that no artificial application is needed when applied. If desired, additional lid liner can be used to color it.

These eye lid adhesive strips can function where they act to cosmetically to augment the other cosmetic make up. Both 303 and 307 can be of different color to match the cosmetic eye makeup and needs of the individuals. The lower and upper lids eye adhesive strip make up can be made up of elastic tissue of synthetic, semi synthetic, plastic, silicone, organic natural material, with pores with different shapes. The pores will help to evaporate the sweat from the eye lids. They are applied to the eye lids with slight stretch which the elasticity exerts slight pressure. It is colored to match the eyes.

The adhesive is attached at the edges or the entire surface of the lids. These Bags under eyes adhesive strip makeup used on the lids can be removed with ease without removing any superficial layers of the skin, and without discomfort. The adhesive should not leave any glue or sticking material on the skin surface of the lids. The undersurface can have therapeutic agents to improve the skin health, anti-wrinkle agents, hormones and various known therapeutic agents, as well as other pharmaceutical, biochemical, nurticeuticals and biological agents or compounds to treat oculopathies including infections, and allergic conditions which at the same coated externally with no reacting and non-allergic cosmetic agents at the same time prevent or treat the condition of bags under eyes. The outer surface is configured to a take with ease any make up applied.

The lid cosmetic bags under the eyes adhesive strips can be supplied on stripping paper which can be cut to the size of the eye lids, peeled, and attached to the eye lids to achieve the desired cosmetic appearance and at the same time prevent bags under the eyes appearing under the lower lids.

The anti-bags under eyes device pockets can be used to deliver known therapeutic agents, other pharmaceutical, biochemical, nurticeuticals, and biological agents or compounds coating or soaking the filling material. Such agents may include but are not limited to tea extracts, hormones, anti-allergy medication, antibacterial, vasoconstrictors, corticosteroids, various medications used to treat oculopathies, antibiotics, nurticeuticals, vitamins, hormones, antiviral, antiwrinkle agents, eye lash growth products, collagen producing agents, growth hormones, dehydrating agents, hyaluronidase, testosterones, estrogens, growth factors and various known various known therapeutic agents, as well as other pharmaceutical, biochemical, nurticeuticals and biological agents or compounds.

The pharmaceutical, biochemical, nurticeuticals and biological agents or compounds enhance the health of the collagen producing cells, skin structure, and musculature under the eye lid skin by using described invention herein. These therapeutic agents can be delivered with peeling type adhesive attached on the back of the pocket facing the eye ball that it can be removed and a new one attached with ease.

The weak electrical stimulator be incorporated under the surface of the bags under the eyes adhesive strips, eye devices, and badges (FIG. 4,5,7, 10,11,12,13) to send peaceable stimulation of electrical current in milliamps to the orbicularis oculi muscle, subcutaneous tissue, and skin of the lower eye lids to strengthen the orbicularis oculi muscle, skin thickness, increase the connective tissue of the dermis, and to hold back the bulging fat and the tissue fluid responsible for the bags under the eyes which hold it in their place in the boney socket under the eye ball where it belongs.

The electromotive force can help absorb the tissue fluid which reduces the size of bags under the eyes. The electronic muscle stimulator produces biphasic square pulse; pulse rate adjustable from 0.5-150 Hz; pulse width and height 50-300 Microseconds; output voltage 0 to 50 V (Load: 1000 ohm); output intensity adjustable, 0-105 milliamps (MA); timer adjustable 1 to 60 minutes or constant.

It is adjustable in 1 minute each step from 1 to 60 minutes. Lead wire can be up to 45 inches long, provided with AC adapter (optional) and belt clip can be incorporated in the stimulator for the eye lids. This electrical stimulator can be used in the treatment of various eye lid diseases and other oculopathies including dry eyes syndrome, age related macular degeneration, retinitis pigmentosa, diabetic retinopathy, and glaucoma.

In another aspect compressor is the transdermal transport of therapeutic agent under the bags under the adhesive strip or eye lid badge aid with therapeutic agent's delivery can be enhanced by the application of a secondary driving force either before or after application of the eye lid adhesive stripe delivery device. In certain aspects this secondary driving force includes electromotive (electrotransport) force in the form of iontophoresis, electroosmosis, electroporation, fractional laser, or mechanical force such as magnetic force, vibration, vibroacoustic force, or sonophoretic force such as ultrasound.

This force may be applied to enhance the uptake of the therapeutic agents into subcutaneous tissue for rapid action and penetration. For example, without wishing to be bound by theory, when ultrasound is used, low frequency is used. In one aspect, less than 2.5 MHz is used. In another aspect, less than 1 MHz is used. When acoustical vibrations (low frequency ultrasound with vibration-vibroacoustic) are applied using a specified pulse ranging, from one-half second to three seconds, modulated with an oscillatory signal in the frequency range of 1 Hz to 1500 Hz, and having pulse amplitude in the range of about 20 to 5000 microns, transdermal transport of a local anesthetic and a therapeutic agent may be further enhanced.

The filling material can be mixed with hot deionized water, gelatin powder, therapeutic plant extracts, hydrogenated castor oil, methylparaben, diazolidinyl urea, a coloring agent, and aroma therapeutic oils. A gelatinous or silicone mould or disc containing various therapeutic agents to treat oculopathies or cosmetic reasons can be placed between the skin of the eye lid and the device which holds it in place to deliver desired therapeutic agents continuously.

The mechanism of action of these inventive devices in preventing, reducing, or eliminating already developed bags under eyes is by physical pressure applied continuously and by electrical and physical methods to strengthen the lower eye lids.

This effect can be augmented by the use of appropriated therapeutic agents/electric stimulator. Due to pressure of the compress on the lower part of the lid and the upper border of the maxilla, the fat and the tissue fluid does not have line of least resistance with gravity to migrate as bags under eyes as it did without the use of our invention (FIGS. 1-15).

When applied to already existing, baggy eyes, the concave semicircular crescent shaped arch of these inventive devices pushes back the content of the bags under the eyes into the orbital cavity tissue space (FIG. 6). The tissue fluid from the bags under eyes is pushed back to the interstitial space where it is picked up by the lymphatic's and the venues due to hydrostatic or physical pressure exerted by pressure of the devices in our invention.

Our test results show that by using the invention for two to three weeks where the invention was applied two to three times during the week the bags under the eyes were reduced in size. These devices can be put on in the night, in the morning, afternoon, and in the evening before going to a dinner or a party that the eyes look cosmetically normal and beautiful. It is best to wear them when working around the house. The eye makeup bags under eyes adhesive strips can be put on at convenience even before attending a party or going to work.

Numerous modifications and alternative arrangements of steps explained and examples given herein may be devised by those skilled in the art without departing from the spirit and the scope of the present invention. The appended claims are intended to cover such modifications and arrangements. Thus, the present invention has been described above with particularity and detail in connection.

The invention will be apparent to those of ordinary skill in the art that numerous modifications, including, but not limited to, variations in size, materials, shape, form function, and manner of procedure, assembly, and the use may be made. The preferred embodiment of the present invention has been described.

The invention should be understood that various changes, adaptations, and modifications may be made thereto. It should be understood, therefore, that the invention is not limited to details of the illustrated invention. This method can be used to diagnose and treat all the oculopathies as well as prevent them. This should be understood, therefore, that the invention is not limited to details of the illustrated invention examples.

Although the instant invention has been described in relation to particular embodiments thereof, many other variations and modifications and other uses will become apparent to those skilled in the art.

What is claimed is:

1. A pair of eyeglasses for treatment of bags under eyes, and swelling and puffiness under the lower eye lids comprising:
a frame having an opening for each eye;
a pocket portion covering a lower portion of said eye openings; and
an insert adapted to fit within said pocket portion wherein said insert is placed in close proximity to a user's face.

2. The pair of eyeglasses for treatment of bags under eyes, and swelling and puffiness under the lower eye lids according to claim 1 wherein said insert is a crescentic badge.

3. The pair of eyeglasses for treatment of bags under eyes, and swelling and puffiness under the lower eye lids according to claim 1 further comprising a medically effective coating on an inward facing portion of said pocket portion.

4. The pair of eyeglasses for treatment of bags under eyes, and swelling and puffiness under the lower eye lids according to claim 1 wherein said insert includes an electrical stimulator for delivering milliamps of electricity to strengthen the lower eye lid muscle and rejuvenate the aging skin of the lower lids and its connective tissue contents.

5. The pair of eyeglasses for treatment of bags under eyes, and swelling and puffiness under the lower eye lids according to claim 1 wherein said pocket portion is made of a flexible material.

6. The pair of eyeglasses for treatment of bags under eyes, and swelling and puffiness under the lower eye lids according to claim 1 wherein said insert is made of a flexible material.

7. The pair of eyeglasses for treatment of bags under eyes, and swelling and puffiness under the lower eye lids according to claim 1 wherein said insert is inflatable to adjust the pressure exerted against said user's face.

8. The pair of eyeglasses for treatment of bags under eyes, and swelling and puffiness under the lower eye lids according to claim 1 further comprising moisture retaining shields disposed on each inner edge of said frame wherein humidity is maintained around the eyes thereby preventing dry eyes.

9. A compressor system for treatment of bags under eyes, and swelling and puffiness under the lower eye lids comprising:
two compressor pockets;
an attachment means for retaining said two compressor pockets against a user's face under each eye; and
an insert removably disposed within each of said two compressor pockets.

10. The compressor system for treatment of bags under eyes, and swelling and puffiness under the lower eye lids according to claim 9 further comprising a bridge disposed between said two compressor pockets wherein said bridge is adapted to rest against a user's nose.

11. The compressor system for treatment of bags under eyes, and swelling and puffiness under the lower eye lids according to claim 9 wherein said attachment means comprises an elastic crescentic shaped adhesive adapted to be applied with slight pressure exerted on Bags under eyes to be pushed back.

12. The compressor system for treatment of bags under eyes, and swelling and puffiness under the lower eye lids according to claim 9 wherein said two compressor pockets are color selectable to match a user's skin color.

13. The compressor system for treatment of bags under eyes, and swelling and puffiness under the lower eye lids according to claim 9 wherein said insert includes an energetic stimulator for delivering milliamps of electricity, vibrator or sonophoresis to strengthen the lower eye lid muscle and rejuvenate the aging skin of the lower lids and its connective tissue contents.

14. The compressor system for treatment of bags under eyes, and swelling and puffiness under the lower eye lids according to claim 9 wherein said attachment means is a self-adhesive portion disposed on an inward surface of said compressor pockets.

15. The compressor system for treatment of bags under eyes, and swelling and puffiness under the lower eye lids according to claim 9 further comprising a medically effective coating on an inward facing portion of said compressor pocket.

16. The compressor system for treatment of bags under eyes, and swelling and puffiness under the lower eye lids according to claim 9 wherein said insert is inflatable to adjust the pressure exerted against said user's face.

17. A method of treating bags under eyes, and swelling and puffiness under the lower eye lids comprising the steps of:
obtaining a pair of eyeglasses for treatment of bags under eyes, and swelling and puffiness under the lower eye lids comprising:
a frame having an opening for each eye;
a pocket portion covering a lower portion of said eye openings; and
an insert adapted to fit within said pocket portion wherein said insert is placed in close proximity to a user's face;
selecting an insert depending on the treatment desired;
placing said pair of eyeglasses on said user's face wherein an inner portion of said pocket portion applies pressure therein; and
wearing said pair of eyeglasses for an effective period of time.

18. The method of treating bags under eyes, and swelling and puffiness under the lower eye lids according to claim 17 further comprising the step of applying a cosmetic coloring to an outside surface of said pocket portions to match said user's skin.

19. The method of treating bags under eyes, and swelling and puffiness under the lower eye lids according to claim 17 further comprising the step of applying a medically effective amount of energy to said user's skin.

20. The method of treating Bags under eyes swelling and puffiness under the lower eye lids complains elastic crescentic shaped adhesive strip according to claim 11 may have different colored lid liner close to the upper and lower eye lid lashes to facilitate eye makeup when using.
